(19) United States
(12) Patent Application Publication
(10) Pub. No.: US 2005/0149081 A1
(43) Pub. Date: Jul. 7, 2005

(54) Endovascular graft and graft trimmer

(76) Inventors: John Ricota, Setauket, NY (US); Benjamin S. Hsiao, Setauket, NY (US); Rajesh H. Somani, Upton, NY (US)

Correspondence Address: DILWORTH & BARRESE, LLP, 333 EARLE OVINGTON BLVD., UNIONDALE, NY, 11553, US

(21) Appl. No.: 10/497,103

(22) PCT Filed: Nov. 27, 2002
(86) PCT No.: PCT/US02/38361

Related U.S. Application Data
(60) Provisional application No. 60/333,920, filed on Nov. 28, 2001.

(51) Int. Cl. A61B 17/32; A61F 2/06

(52) U.S. Cl. 606/159; 623/1.15; 623/1.32; 623/1.35; 606/170

(57) ABSTRACT

An endovascular graft includes a flexible graft body dimensioned for positioning within a body vessel and having an outer wall defining an opening therethrough, and an elastic polymer strip mounted to the outer wall of the graft body. The elastic strip is dimensioned to increase an effective strength of the outer wall to thereby facilitate support of the body vessel upon implantation of the graft body therewithin. The graft body is preferably bifurcated including a tubular body defining a longitudinal opening and first and second legs connected to the tubular body. The first and second graft legs each define a longitudinal opening in communication with the longitudinal opening of the tubular body. The tubular body and the first and second legs preferably include the elastic polymer strip. The polymer strip is dimensioned to extend substantially along the lengths of the respective tubular body and the first and second legs. The graft elastic polymer strip may be helically disposed relative to the graft body. The graft elastic polymer strip may comprise a biodegradable polymer. Suitable polymers include may be selected from the group consisting of polyglyocolide, polylactide, and copolymers of glycolide and lactide. The graft body may include two sheets of graft material joined together with elastic polymer strip disposed between the two sheets of graft material. The elastic polymer strip may includes vascular medication for treatment of the patient. Preferably, a plurality of elastic polymer strips helically disposed relative to the graft body.

### CROSS REFERENCE TO RELATED APPLICATIONS

This application is based on and claims priority to U.S. Provisional Application No. 60/333,920 filed Nov. 28, 2001.

### FIELD OF INVENTION

The present invention relates to endovascular grafts, and related apparatus and method for application of the endovascular grafts within body vessels, such as abdominal aneurysms.

### BACKGROUND OF THE INVENTION

Endovascular grafts have been developed to treat patients with arterial lesions, particularly, aneurysms, trauma and arterial dissections, from within the arterial tract to reduce morbidity and mortality associated with the arterial disorder. Application of the graft is typically performed in conjunction with a minimally invasive operative procedure to minimize patient trauma, recovery time, etc. A variety of endovascular grafts or stent-grafts are currently on the market or in clinical trials. These grafts have a number of different characteristics related to their construction, support with respect to the vessel wall and fixation mechanisms. Current endovascular grafts generally consist of a fabric graft (such as Teflon or Dacron, etc.) and metallic stents. The metallic stents are used to support the graft at the proximal and distal attachment sites, or throughout the length of the graft. Fixation of the endovascular graft can be generally achieved through radial wall tension using a self-expanding metallic stent or by balloon expansion of a deformable, metallic stent which may possess fixation elements to penetrate the arterial wall.

One example of such endovascular graft is disclosed in U.S. Pat. No. 5,667,523 issued to Bynon et al. The '523 graft is a dual supported vascular graft including a biocompatible flexible layer sandwiched between two balloon expandable stents.

Endovascular grafts with bifurcated configuration have been currently introduced for treatment of abdominal aortic aneurysms. Many bifurcated grafts are of a two piece design. These two piece designs require the insertion of a contralateral limb through a separate access site. These types of grafts are complex to deploy and have the potential for leakage at the connection site of the two limbs of the graft. One piece bifurcated grafts have also been designed. However, their deployment is still somewhat complicated and has torsion tendencies.

One piece bifurcated grafts are well known in the art. For example, U.S. Pat. No. 2,845,959 discloses a one-piece seamless woven textile bifurcated tube for use as an artificial artery. Yarns of varying materials can be used to weave the bifurcated graft including nylon and plastic yarns. U.S. Pat. Nos. 3,096,560 and 3,029,819 issued to Liebig and Starks, respectively, disclose woven one-piece bifurcated grafts which are constructed by performing specific types of winding and weaving about a smooth bifurcated mandrel.

U.S. Pat. No. 4,497,074 describes a one-piece bifurcated graft which is made from a preformed support in the shape of the bifurcated graft (i.e. mould). In a first stage, a gel enabling a surface state close to that of the liquid-air interface to be obtained at the gel-air interface is deposited by dipping or coating the preform with a sol which is allowed to cool. A hardenable flexible material such as a silicone elastomer by dipping or spraying the material on the mould in a second stage. Finally, after hardening of the material, the prosthesis is removed from the mould. In U.S. Pat. No. 4,816,028 issued to Kapadia et al., there is shown a one-piece woven bifurcated vascular graft having a plurality of warp threads running in the axial direction and a plurality of weft threads running in the transverse direction. Further, U.S. Pat. No. 5,108,424 issued to Hoffman, Jr. et al. discloses a one-piece bifurcated collagen-impregnated Dacron graft. The bifurcated graft includes a porous synthetic vascular graft substrate formed by knitting or weaving with at least three applications of dispersed collagen fibrils.

The Herweck et al. patent, U.S. Pat. No. 5,197,976, discloses a continuous one-piece bifurcated graft having plural longitudinally parallel tube structures which are attached to one another over at least a portion of their longitudinal exteriors. The tube structures can be manually separated to form a branched tubular structure. The prosthesis is manufactured by paste forming and stretching and/or expanding highly crystalline unsintered polytetrafluoroethylene (PTFE). Paste forming includes mixing the PTFE resin with a lubricant, such as mineral spirits, and then forming the resin by extrusion into shaped articles.

Although the above-described one-piece bifurcated grafts have addressed leakage and graft failure at the suture or juncture site associated with two-piece bifurcated grafts which form the bifurcated graft, problems still exist with these one-piece bifurcated grafts. For example, the prior art bifurcated grafts do not include an integral support structure to restrain from deformation, twisting or collapse of the graft limbs. Further, graft migration problems are still prevalent.

### SUMMARY OF THE INVENTION

Accordingly, the present invention is directed to endovascular grafts, related apparatus and method for applying such grafts with body vessels, such as abdominal aneurysms. In one preferred embodiment, a graft for positioning within a body vessel includes a flexible graft body dimensioned for positioning within a body vessel and having an outer wall defining an opening therethrough, and an elastic polymer strip mounted to the outer wall of the graft body. The elastic strip is dimensioned to increase an effective strength of the outer wall to thereby facilitate support of the body vessel upon implantation of the graft body therewithin. The graft body is preferably bifurcated including a tubular body defining a longitudinal opening and first and second legs connected to the tubular body. The first and second graft legs each define a longitudinal opening in communication with the longitudinal opening of the tubular body. The tubular body and the first and second legs preferably include the elastic polymer strip. The polymer strip is dimensioned to extend substantially along the lengths of the respective tubular body and the first and second legs. The graft elastic polymer strip may be helically disposed relative to the graft body. The graft elastic polymer strip may comprise a biodegradable polymer. Suitable polymers include may be selected from the group consisting of polyglyocolide, polylactide, and copolymers of glycolide and lactide. The graft body may include two sheets of graft material joined together with elastic polymer strip disposed between the two sheets of graft material. The elastic polymer strip may include vascular medication for treatment of the patient. Preferably, a plurality of elastic polymer strips helically disposed relative to the graft body. The elastic polymer strips may be arranged in a cross-hatched lattice network configuration. A detachable string mesh may be disposed about the graft body for facilitating retention of the graft body in a compressed state.

A graft cutter adapted to trim a vascular graft subsequent to placement of the vascular graft in a vascular organ is also disclosed. The graft cutter includes a rotatable ring and a plurality of blades operatively connected to the rotatable ring and movable between an open position for receiving the vascular graft and a closed position for cutting the vascular graft upon rotation of the rotatable ring. Alternatively, the graft cutter may include a lasso cutter having a sharp wire-like material and adapted for movement between an open configuration for receiving the vascular graft and a closed configuration for compressing against and thereby cutting the vascular graft.

A method of deploying a bifurcated vascular graft within an abdominal aneurysm is also disclosed.

### BRIEF DESCRIPTION OF THE DRAWINGS

Preferred embodiments of the present invention are described herein with reference to the drawings wherein:

FIG. 1 is a partial cross-sectional view illustrating the state of the bifurcated graft of the present invention deployed within the vessels, e.g., abdominal aorta and iliac arteries;

FIG. 2 is a perspective view of the bifurcated graft constructed in accordance with the principles of the present invention;

FIG. 3 is an elevational view of two overlapping graft sheets (with two helix network) before formed into a tubular graft body of the present invention;

FIG. 3A is an enlarged cross-sectional view of the graft structure, taken along the line A-A in FIG. 3;

FIG. 4 is a perspective view illustrating one example of the graft structure including two helices network of flexible polymer rods;

FIG. 5 is an elevational view of two overlapping graft sheets (with one helix network) before formed into a tubular graft body of the present invention;

FIG. 6 is a perspective view illustrating another example of the graft structure formed from the two graft sheets of FIG. 5;

FIG. 7 is an elevational view of two overlapping graft sheets (with three helix network) before formed into a tubular graft body of the present invention;

FIG. 8 is a perspective view illustrating another example of the graft structure formed from the two graft sheets of FIG. 7;

FIG. 9 is an elevational view of two overlapping graft sheets (with four helix network) before formed into a tubular graft body of the present invention;

FIG. 10 is a perspective view illustrating another example of the graft structure formed from the two graft sheets of FIG. 9;

FIG. 11 is a schematic view of the graft cutter of the present invention;

FIG. 12 is a schematic view illustrating alternate embodiment of the graft cutter of the present invention;

FIG. 13 is a partial cross-sectional perspective view illustrating the deployment apparatus with the graft of the present invention mounted inside the outer sheath of the apparatus;

FIG. 14 is a partially exploded perspective view illustrating the bifurcated graft of the present invention with mesh wrap outside the graft body and the first and second graft legs, which is positioned inside the deployment device;

FIG. 15 is a partial cross-sectional view illustrating positioning of the deployment catheter containing the bifurcated graft inside the blood vessel, e.g., thoracic aorta;

FIG. 16 is a view similar to the view of FIG. 15 illustrating the outer sheath retracted from the graft, while both the body and legs still remaining wrapped by the wire mesh;

FIG. 17 is a view similar to the view of FIG. 15 illustrating positioning of the graft using the catheter from the iliac artery;

FIG. 18 is a view similar to the view of FIG. 15 illustrating deployment of the graft body (through self-expansion to conform to the vessel diameter), while the two legs still remaining inside the mesh wrap;

FIGS. 18A and 18B are views similar to the view of FIG. 18 illustrating further deployment of the two graft legs in sequence;

FIG. 19 is a partial cross-sectional view illustrating positioning of the hybrid stent-graft of the present invention inside the blood vessel;

FIGS. 20 and 20A are views similar to the view of FIG. 19 illustrating deployment of the hybrid stent-graft inside the vessel;

FIG. 21 is a partial cross-sectional view illustrating the enodluminal sight of the visceral vessels;

FIG. 22 is a view similar to the view of FIG. 21 illustrating a fenestrated module deployed within the blood vessel;

FIG. 23 is a view similar to the view of FIG. 21 illustrating deployment of two overlapping fenestrated modules with oval openings arranged towards visceral vessels; and

FIG. 24 is a view similar to the view of FIG. 21 illustrating fixation of the fenestrated modules using endovascular staples.

### DETAILED DESCRIPTION OF THE PREFERRED EMBODIMENTS

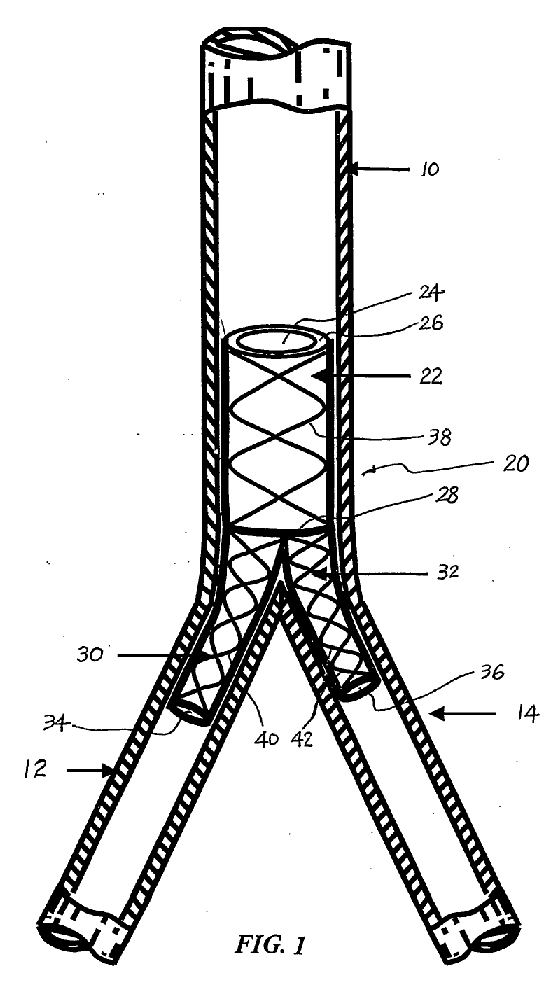

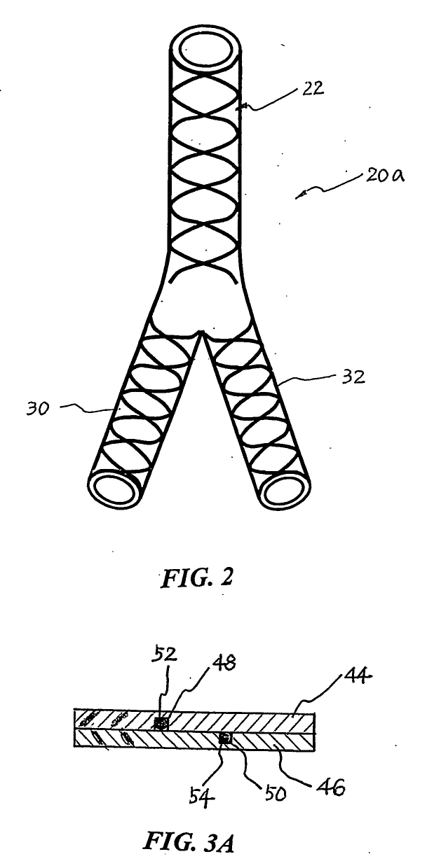

Referring now to the drawings in which like reference numerals indicate similar or identical elements throughout the views, FIG. 1 illustrates the bifurcated vascular graft constructed in accordance with the principles of the present invention, which is deployed within the blood vessels including aorta 10 femoral artery 12 and iliac artery 14. Bifurcated vascular graft 20 includes a flexible tubular graft body 22 having a longitudinal opening 24, top and bottom ends 26, 28. The graft 20 further includes first and second flexible tubular graft legs 30, 32 joined longitudinally to the bottom ends 28 of the graft body 22 in a sealing relation therebetween. Each graft legs 30, 32 includes longitudinal opening 34, 36 in communication with the longitudinal opening 24 of the graft body 22. The graft body 22 and the first and second graft legs 30, 32 include at least one set of elastic polymer strips 38, 40, 42 disposed respectively to the graft body 22 and the graft legs 30, 32 substantially along the lengths thereof. The strips 38, 40, 42 are preferably disposed helically within the graft body and legs. Graft body 22 and graft legs 30, 32 can be formed from separate parts joined together as illustrated in FIG. 1, or by a single part as illustrated in FIG. 2. In either structure, the vascular graft 20 or 20a works as a unibody incorporating a graft body 22 and two graft legs 30, 32 in an inverted Y fashion. The length of graft body is preferably about 2 to 5 cm, and that of the legs 30, 32 can be varied depending upon the surgical needs. The ratio of the diameter of the body 22 to that of the legs 30, 32 is about 2 to 1. Graft body 22 and graft legs 30, 32 are formed from a substantially flexible polymer material such as polytetraluorethylene (PTFE) or Dacron® (polyester). Other flexible or stretchable materials of biocompatible characteristics can be used for the graft material. Elastic polymer strips 38, 40, 42 are formed from a flexible or elastic biodegradable polymer material, such as polyglyocolide, polylactide, and copolymers of glycolide and lactide. It is noted that polymer strips 38, 40, 42 have in addition to elasticity, a suitable stiffness or strength which is greater than that of the graft material to provide necessary support against the inner walls of the blood vessels. The polymer strips are preferably arranged in a helical, sinusoidal or cross-hatched lattice network to serve as a self-expanding stent to support the vascular graft 20. The polymer can be formed or molded in the form of a thin rod or rectangular strip of suitable dimensions and provides sufficient flexibility and stiffness to the graft.

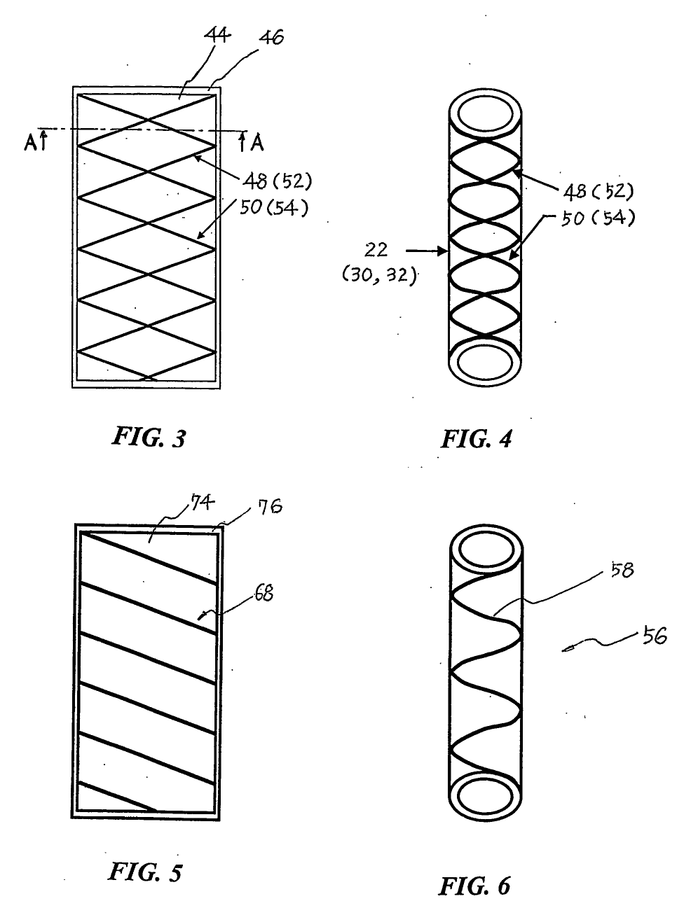

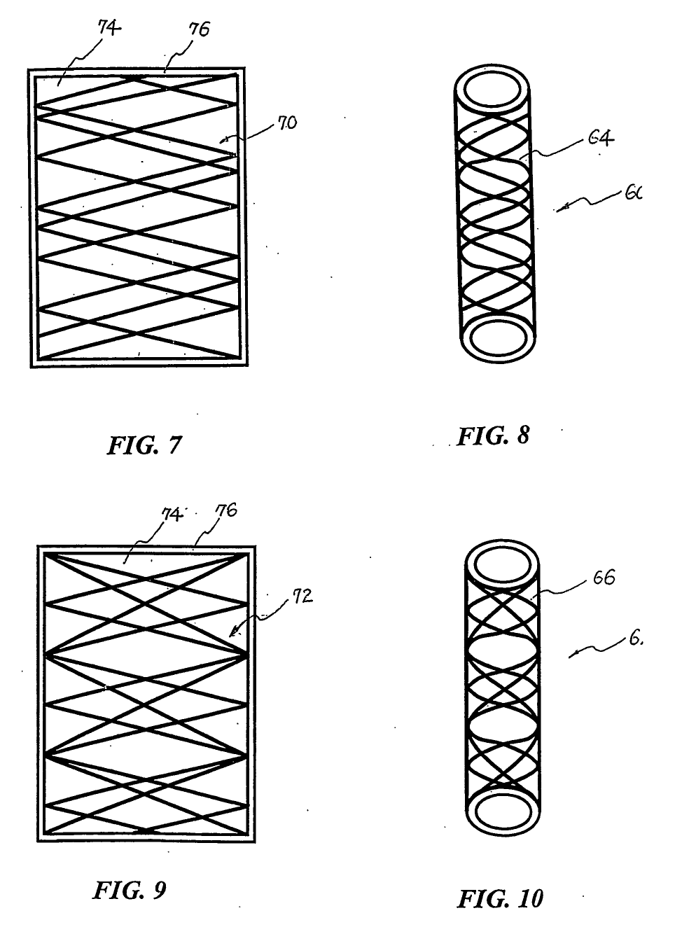

With reference to FIGS. 3-10, preferable embodiments of the graft structure and method of making the graft (such as graft body 22 and graft legs 30, 32 of FIG. 1) are herein discussed. Initially, an outline of suitable lattice network is drawn and formed on each opposing surface of two rectangular graft sheets 44, 46, respectively. FIG. 3 illustrates one preferred embodiment of the graft structure in that two sets of parallel straight grooves 48 and 50 are formed on the two grafts, i.e., grooves 48 formed on the first graft 44 and grooves 50 formed on the second graft 46, respectively. FIG. 3A illustrates a cross-sectional view of the graft structure. The graft sheets 44, 46 are made of substantially flexible graft material, such as PTFE, polyester, etc. The outline can be preferably configured as a cross-hatched lattice network as shown in FIG. 3. The two graft sheets 44 and 46 are then joined together using a variety of techniques, such as heat press, sewing, etc. The two sheets are joined except along the outlines, previously formed on the graft, thus leaving a hollow gap at the outlines between the graft sheets. A plurality of biodegradable polymer rods or strips 52 and 54 are inserted inside the hollow gaps. Alternatively, the two sheets 44 and 46 can be joined after insertion of the polymer strips 52 and 54 to the hollow grooves 48 and 50 of the sheets. FIG. 3A shows an enlarged cross-sectional view of the graft sheets 44 and 46 with polymer strips 52 and 54 assembled therein to their respective grooves. The two sheets containing the polymer strips are then rolled to form a tubular configuration and joined at the corresponding lateral sides with a suitable joining method such as bonding by adhesive, sewing, etc. An exemplary graft component, such as graft body 22 and graft legs 30, 32 formed in accordance with the principles of the invention is illustrated in FIG. 4. It is noted that the overall strength, such as stiffness and flexibility, of the cylindrical graft, when formed in a cylindrical tube, can be controlled by selecting the size(e.g., polymer strip diameter) and/or pitch of the helical network pattern.

FIGS. 6, 8, 10 illustrate alternate embodiments of the graft structure of the present invention. The structure and method of manufacturing such grafts are substantially similar to that of FIG. 3 described herein above, except they differs in the number and configuration of the helical networks of polymer strips formed on the graft. FIG. 6 illustrates a graft structure 56 where a single helix of polymer strips 58 are formed therein. Likewise, FIGS. 8 and 10 illustrate alternating graft structure 60 and 62 where three and four helix of polymer strips 64 and 66 are respectively formed therein. Preferable method of making such grafts are generally similar to that described herein-above in connection with FIG. 3, however, the network outlines are different. FIGS. 5, 7, 9 illustrate such exemplary network outlines 68, 70, 72, respectively formed in two graft sheets 74 and 76, in order to respectively make the grafts of FIGS. 6, 8, 10 by rolling and joining in the manner described above. Other suitable network configurations can be similarly contemplated.

The overall strength of the graft can be optimized by selecting suitable strip size, pattern and/or the number of the polymer helix. Such flexible or elastic polymer strips arranged in a suitable fashion on the body and legs of the graft will allow desired flexibility for deployment of the graft and necessary support when it was fixed within the body vessels. The graft can be compressed to a small size when it is carried or delivered within the body vessels utilizing suitable surgical technologies known in the art, such as carrying within a delivery sheath. After delivered to the site, the graft can be then expanded to provide the desired frictional support simply by releasing from compressed state.

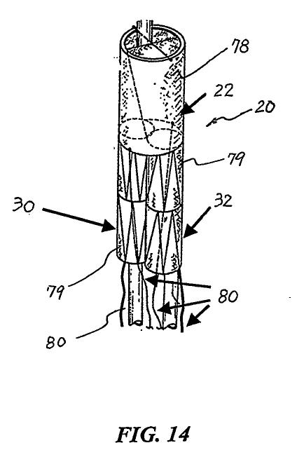

The body and each leg of the graft are preferably wrapped separately in detachable or removable mesh wraps 78, 79 (FIG. 14). In addition, the entire graft may be wrapped in a second outer external wrap (not shown) for delivery. The graft may preferably be delivered from within a delivery sheath of suitable deployment apparatus (to be discussed later in detail), in which case the outer wrap is not required. This design allows the graft body 22 to be deployed independent of the graft legs 30, 32. Alternatively, the graft can delivered bare, i.e., without having any external wraps, over a guiding catheter that goes through the longitudinal openings, such as openings 34 and 24 of one graft leg 30 and graft body 22 of the graft. The proximal and distal attachment sites may be fixed with endovascular staples to help secure the graft. Examples of such staples and related stapler are disclosed in a previous Provisional Patent Application Ser. No. 60/285,101 of the common applicants, the contents of which are incorporated herein by reference. Each legs 30 or 32 of the graft has a series of strings 80 attached adjacent the circumference of the graft in a parachute-like configuration to allow the legs to be manipulated.

The surface of the polymer strips can be coated, or otherwise blended inside the bulk matrix of the polymer, with suitable vascular medication, thus can provide with healing and drug release functions. In a recent article¹ use of a biodegradable polymer for drug delivery has been described. The basic requirement for synthetic polymeric delivery systems is that they be free from potential carcinogenicity or unexpected inflammatory reaction due to cytotoxicity or immunogenicity when implanted into humans. The biodegradable polymer, poly-D,L-lactic acid-dioxanone olyethylene glycol block copolymer (PLA-DX-PEG polymer) and its degradation products are thought to meet those demands because PLA, DX, and PEG homopolymers have been shown to be compatible and safe for clinical use through their use as suture materials or in other drug delivery systems. Linear aliphatic polyesters such as polyglycolide (PGA), or its random copolymer poly (glycolide-co-lactide) (PGA-co-PLA) are also bioabsorbable and biodegradable. Also, the copolymer exhibits good biocompatibility and biodegradability properties. The great advantage of these synthetic absorbable materials is due to their degradability by simple hydrolysis of their ester backbone in aqueous environments, such as body fluids. The degradation products are ultimately metabolized to carbon dioxide and water or can be excreted via the kidney. It is also known in the art that the degradation rate of these materials can be controlled by varying the molecular structure, molecular weight, processing parameters and morphology. These polymers can be molded in the form of rods or strips (with the drug blended inside the bulk matrix of the polymer) using the standard polymer molding equipment. Thus, the polyglycolide, polylactic acid and their copolymers in the form of helical lattice network of the graft (disclosed here) can provide a reliable drug delivery system, although further tests in large animals or primates will be essential before it can proceed to clinical applications.
Naoto Saito, Takao Okada, Hiroshi Horiuchi, Narumichi Murakami, Jun Takahashi, Masashi Nawata, Hiroshi Ota, Kazutoshi Nozaki, and Kunio Takaoka, “A biodegradable polymer as a cytokine delivery system for inducing bone formation”, Nature Biotechnology, 19, Apr. 2001, 332.

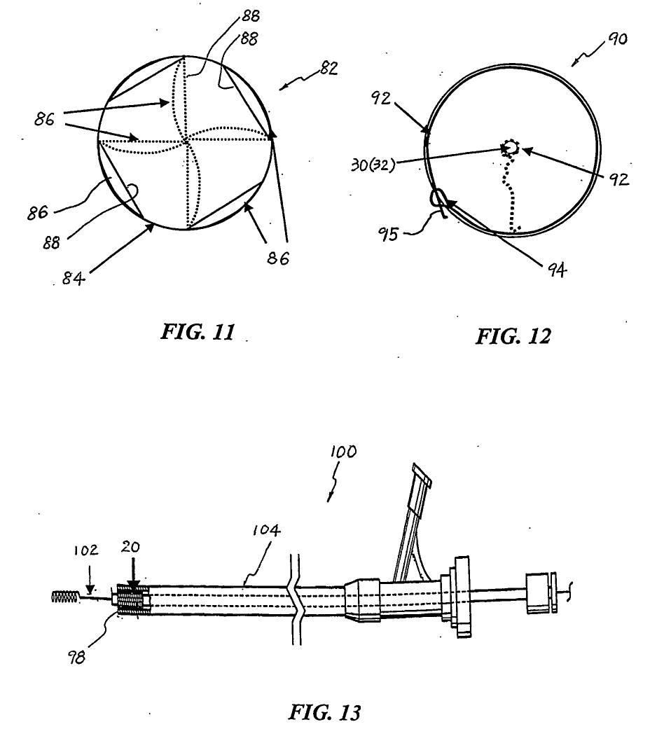

With reference to FIGS. 11 and 12, preferred embodiments of graft cutter or trimmer for trimming an endograft within the vessels at the time of deployment is discussed herein. The device is low profile and flexible. One preferred embodiment of the graft cutter mechanism is illustrated in FIG. 11. The graft cutter 82 includes a rotatable ring 84 and a plurality of circumferential blades 86 operatively connected to the rotatable ring 84 to provide an open position and a closed position for cutting the graft introduced therein. The ring 84 is placed over the graft and rotating the device so that the blades 86 close like the shutters of a camera and cut the graft. FIG. 14 shows the blades in an open position (shown in solid lines) for receiving the graft, while a closed position is illustrated in phantom lines where sharp edges 88 of the blades complete cutting action to the graft. A miniature electric coil (not shown) can be attached to the cutter blades 86 and a small electrical current (e.g. monopolar cautery) may be applied when necessary to facilitate cutting of the graft.

Another embodiment of the graft cutter is schematically illustrated in FIG. 12. This cutter is designed as a lasso cutter where the lasso placed over the graft exits from a sheath and is tightened by pulling against the sheath. The lasso cutter 90 has sharp (and thin) metal wire 92 adapted to enclose the graft and loop 94 for receiving a free end 95 of the wire 92 therethrough for providing the tightening motion. By pulling the free end 95 of the metal wire blade 92 introduced within the loop 94, the wire blade 92 compresses inward and ultimately cuts the graft such as graft legs 30 and 32. Such compressed or closed position for cutting is shown in phantom. Again, a miniature electrical coil (not shown) can be attached to the lasso wire and monopolar cautery may be used to facilitate the graft cutting.

Application for Treating Abdominal Aorta Aneurysms:

With reference now to FIG. 13, an exemplary deployment apparatus for deployment of the grafts of the present invention is discussed herein. Devices for deployment of one-piece or unibody bifurcated grafts are available and well known in the art. One such apparatus is disclosed in U.S. Pat. No. 6,210,422, the entire contents of which are incorporated herein by reference. The 22 apparatus incorporates a first and second hollow limb tubes for containing the legs of the bifurcated graft, a third hollow tube for containing the body of the graft, and a hollow delivery tube capable of encompassing the limb tubes and graft body tube. A similar device can be used for introducing the graft of the present invention in the blood vessels.

As shown in FIG. 13, bifurcated graft 20 of the present invention is collapsed to a compressed state, and then inserted into the distal hollow cavity of outer sheath 98 of deployment apparatus 100 for the delivery and positioning of the graft Into the blood vessels. Deployment apparatus 100 preferably includes guide wire 102 to facilitate navigation of catheter 104 of the deployment apparatus into the vessels. FIG. 14 shows a perspective view of the graft 20 with graft body 22 and graft legs 30, 32 collapsed and wrapped by removable mesh 78, 79, and further introduced within the outer sheath (not shown) before deployment.

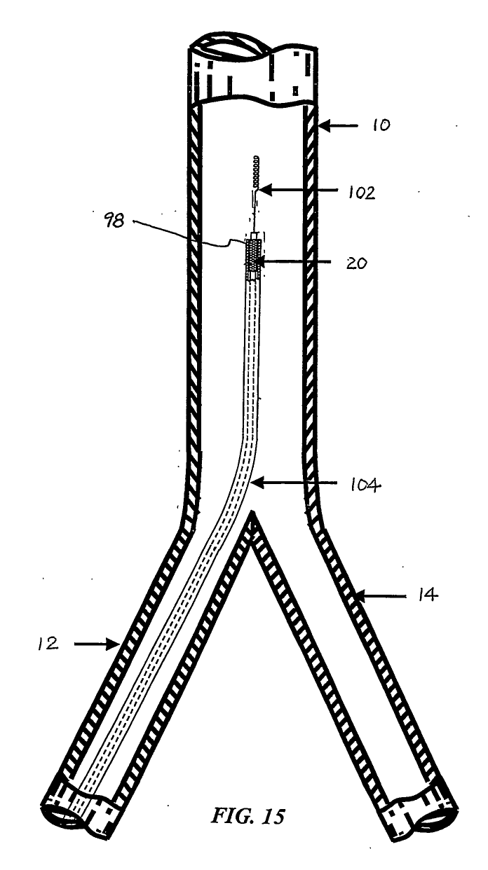

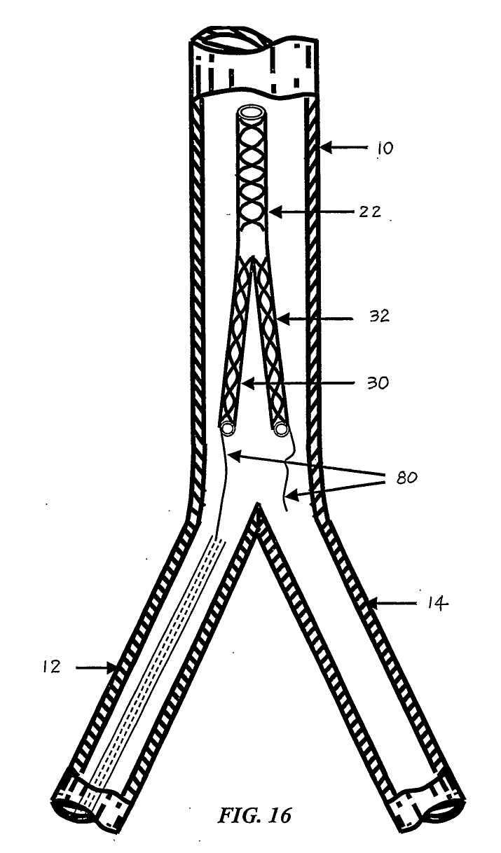

With reference to FIG. 15 in association with FIG. 16, deployment catheter 104 of the deployment apparatus 100 is introduced into thoracic aorta 10 through femoral artery 12. Delivery sheath 98 is now removed from the graft 20 (FIG. 16). Alternatively, as a delivery option described herein-above in connection with the description of the graft, if the graft is directly mounted over the delivery catheter for the delivery (i.e., when the delivery sheath is not used for receiving the graft therein), additional outer wrap is instead removed from the graft.

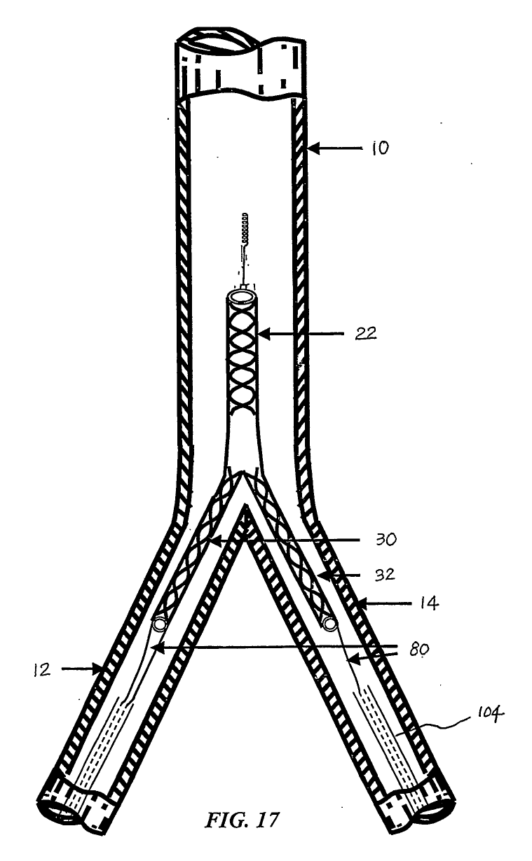

Referring now to FIG. 17, using catheters 104 from the contra-lateral femoral artery, parachute strings 80 attached to the contra-lateral legs are grasped and delivered into the contra-lateral iliac artery 14. The graft is still compressed by wire mesh 78 and 79 at this point. The graft body 22 is then positioned below the renal arteries.

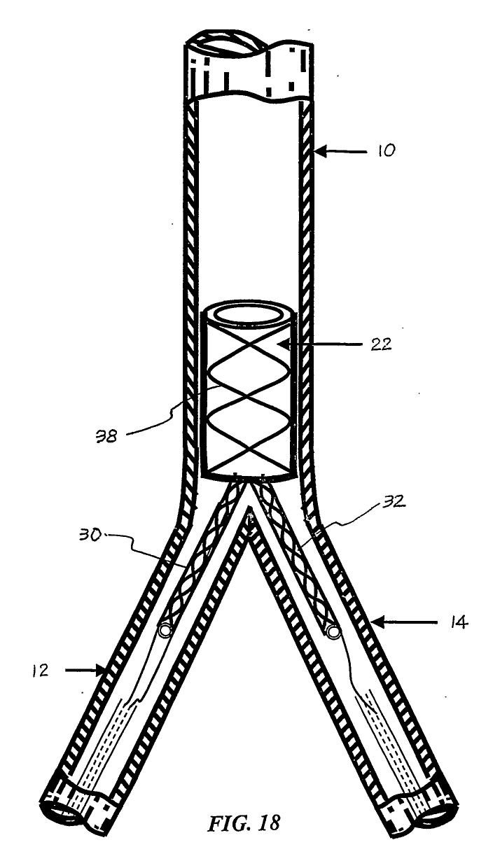

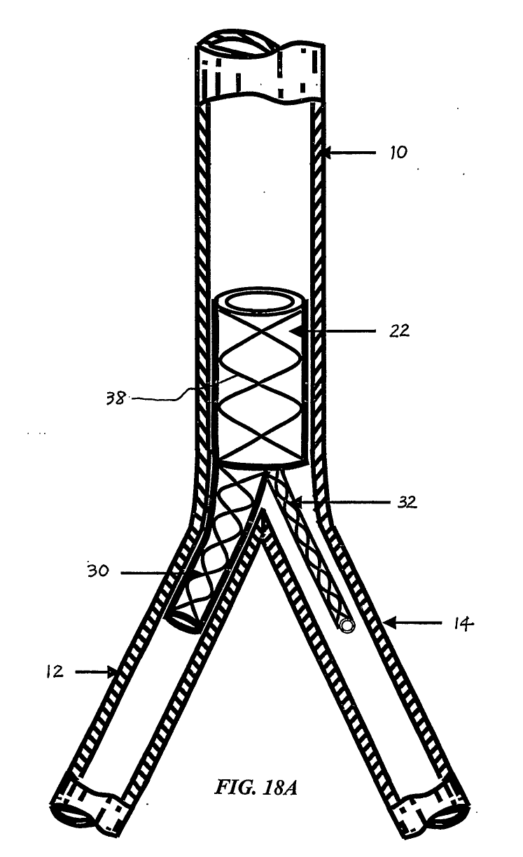

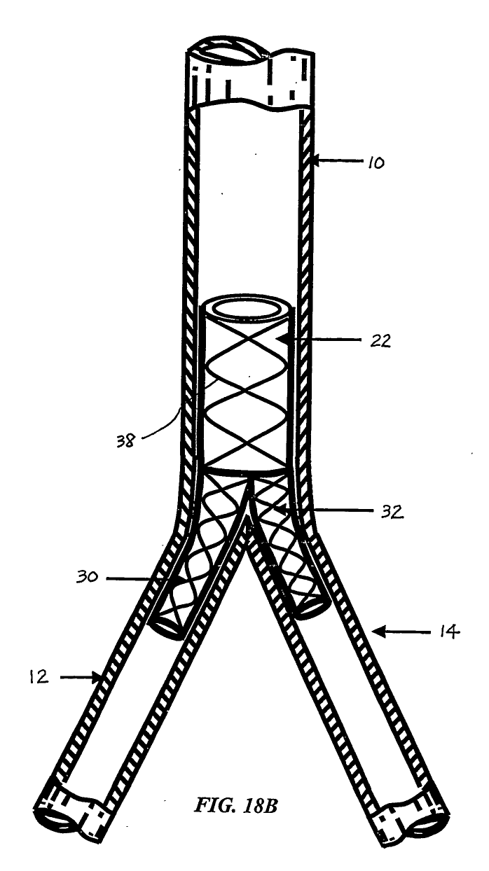

With reference to FIG. 18, the graft body 22 is deployed by removing the outer wrap or mesh 78 that constrains the self-expanding biodegradable stents 38 therein. Once the graft body 22 is deployed, it is held in place by the biodegradable stent 38 in its expanded, non-compressed state. Both graft legs 30 and 32 are still inside wire mesh 79. A flexible delivery sheath may then be placed over each graft leg through the corresponding femoral artery. A suitable graft cutter, such as graft trimmer 82 or 90 (FIGS. 11, 12) is then placed within the sheath over each graft legs, which continues to be contained in its external wrap. The length of the graft leg is determined under fluoroscopic control by positioning the sheath and applying traction on the parachute wires 80. The sheath is withdrawn to 1-2 cm above the distal landing zone and the external sheath constraining the legs 30 and 32 is removed allowing the legs to expand. At this point, final adjustments to the distal landing site are made and the graft is cut using the graft trimmer, e.g., 82 or 90. Cutting the graft with the graft trimmer releases the last segment of the external wrap, and when the delivery sheath is removed each graft leg 30, 32 expands and deploys to its position by the self-expanding stents 38. FIGS. 18A and 18B illustrate sequential views of deployment of such graft legs respectively within the femoral artery 12 and the iliac artery 14.

Following the deployment of the entire graft, the graft may be further fixed by balloon expansion of known type (not shown). Finally, one or more rings of endovascular staples can be used to fixate the proximal and distal attachment sites. One example of such endovascular staple device is disclosed in commonly assigned Provisional Patent Application Ser. No. 60/285,101, the contents of which are incorporated herein by reference. A similar design can be used to place proximal and distal extensions if required, and the fixation points of the extensions can be likewise secured with such endovascular staples.

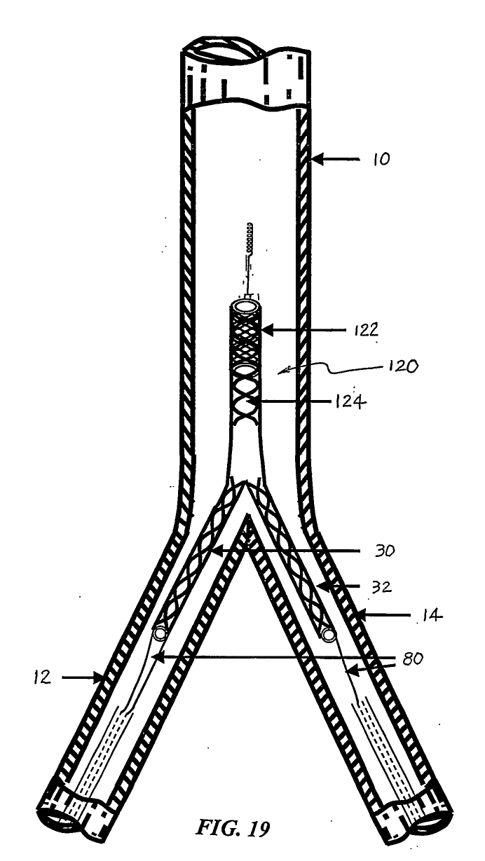

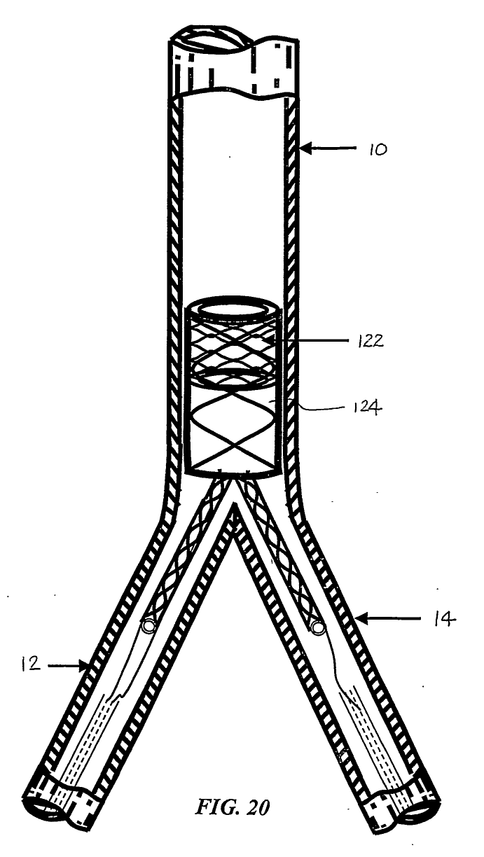

Referring to FIGS. 19 and 20, deployment of a hybrid bifurcated graft of the present invention is discussed herein. Hybrid graft 120 includes a self-expanding stent or stent-graft 122 connected to the top end of a graft 124 which is similar to the graft 20 discussed above. The self-expanding stent 122 provides necessary support for the graft during positioning inside the body vessel as well as after deployment. The stent 122 has similar structure and configuration as to the graft 124, which includes a flexible graft body and elastic polymer strips. Overall strength of the stent 122 is preferably higher than the graft 124. Alternatively, stent 122 may include self-expanding metallic strips in lieu of, or in combination with, the elastic polymer strips. Such metallic strips are known in the art. The hybrid graft 120 can be introduced and deployed within thoracic aorta 10 in a manner similar to that described herein-above. FIG. 19 shows the hybrid stent/graft 120 inside the vessel before deployment, and FIGS. 20 and 20A show sequential views of deployment of the stent/graft 120 and the graft legs 30, 32 respectively within the femoral artery 12 and the iliac artery 14.

Application for Treating Suprarenal and Thoraco Abdominal Aneurysms:

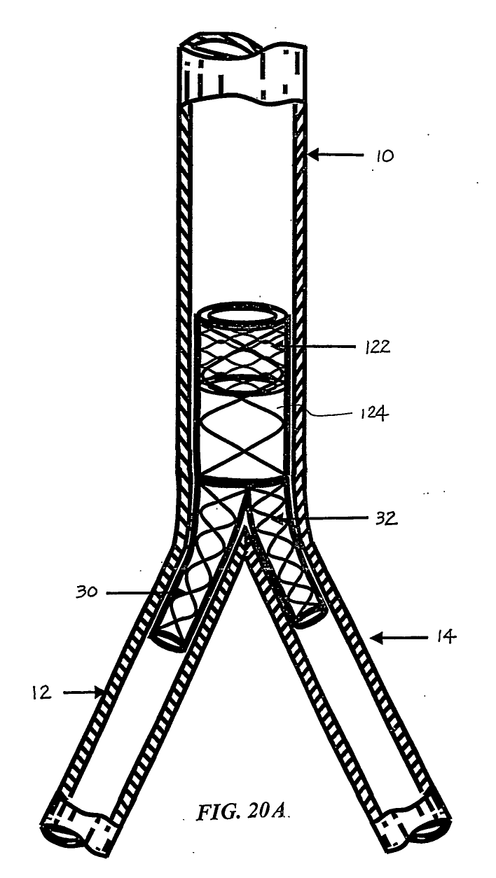

Employing a modular concept, endovascular grafts of the invention can be used to treat thoracic or thoraco-abdominal aneurysms. The major problem with these aneurysms is maintenance of visceral perfusion. In accordance with the principles of the present invention discussed herein, with reference to FIGS. 22 and 23, three different types of modular grafts can be used for this purpose. The first is the standard tube or bifurcated graft 136, which is used to cover the proximal and distal extent of the aneurysm. The second module is “fenestrated modules” 138, 142 which consist of a tube graft with an oval fenestration in it which is placed to maintain perfusion to the visceral vessels. By overlapping two fenestrated modules 138, 142, the size of the opening to the visceral vessels can be controlled. These modules will need to be custom designed so that the dimensions of the ellipse (opening) will be adequate for the visceral “patch” of an individual patient. The final modular piece can be the “plug” (not shown). This is a straight tube with a flared end, which is designed to go into the orifice of a visceral vessel and then have the “flare” for a seal with “endovascular staples”.

With reference to FIGS. 21-24, deployment of a graft of the present invention for treating thoracic or thoraco-abdominal aneurysms is discussed herein. FIG. 21 illustrates an enodoluminal view of a thoracic artery 130 and the visceral vessels, such as a celiac artery 132 and renal artery 134. As a first step, a modular straight graft 136 is deployed from the origin of the aneurysm to 2-3 cm above the origin of the celiac axis. A first fenestrated module 138 with its oval hole 140 directed towards the origin of the visceral vessels is then deployed overlapping the initial module so that the upper portion of the hole 140 is positioned just above the origin of the celiac artery 132 (FIG. 22). A second fenestrated module 142 is deployed so that the lower portion of the oval hole 140 is just below the origin of the renal arteries (FIG. 23). By overlapping these two fenestrated modules 138 and 142, an orifice for the visceral vessels is created. The modules can be connected using endovascular staples 144 in the similar manner discussed above under fluoroscopic or IVUS control (FIG. 24). If necessary a “plug” module (not shown) is inserted which is directed into the orifice of the visceral vessel in question to seal any leaks.

While the above description contains many specifics, these specifics should not be construed as limitations on the scope of the disclosure, but merely as exemplifications of preferred embodiments thereof. Those skilled in the art will envision many other possible variations that are within the scope and spirit of the invention.

1. A graft for positioning within a body vessel, which comprises:
a flexible graft body dimensioned for positoning within a body vessel, said graft body including an outer wall defining an opening therethrough; and
an elastic polymer strip mounted to said outer wall of said graft body, said elastic strip dimensioned to increase an effective strength of the outer wall to thereby facilitate support of the body vessel upon implantation of said graft body therewithin.

2. The graft of claim 1 wherein said graft body includes a tubular body defining a longitudinal opening and first and second legs connected to said tubular body, said first and second graft legs each defining a longitudinal opening in communication with said longitudinal opening of said tubular body.

3. The graft of claim 2 wherein each said tubular body and said first and second legs include said elastic polymer strip.

4. The graft of claim 3 wherein each said polymer strip is dimensioned to extend substantially along the lengths of said respective tubular body and first and second legs.

5. The graft of claim 1 wherein said elastic polymer strip is helically disposed relative to said graft body.

6. The graft of claim 1 wherein the elastic polymer strip comprises a biodegradable polymer.

7. The graft of claim 6 wherein said biodegradable polymer is selected from the group consisting of polyglyocolide, polylactide, and copolymers of glycolide and lactide.

8. The graft of claim 1 wherein said graft body includes two sheets of graft material joined together, and said elastic polymer strip is respectively disposed between said two sheets of graft material.

9. The graft of claim 1 wherein said elastic polymer strip includes vascular medication for treatment of the patient.

10. The graft of claim 5 including a plurality of said elastic polymer strips helically disposed relative to said graft body.

11. The graft of claim 10 wherein said elastic polymer strips are arranged in a cross-hatched lattice network configuration.

12. The graft of claim 1 further including a detachable string mesh disposed about said graft body for facilitating retention of said graft body in a compressed state.

13. The graft of claim 1 further including a self-expanding stent connected to at least one distal end of said graft body.

14. The graft of claim 13 wherein said self-expanding stent includes an elastic polymer strip to increase the strength of the graft.

15. The graft of claim 13 wherein said self-expanding stent includes a metallic strip to increase the strength of the graft.

16. The graft of claim 1 wherein the outer wall of said graft body includes a fenestration.

17. The graft of claim 16 wherein the fenestration of the graft body is dimensioned to encircle visceral vessels of a patient.

18. A graft cutter adapted to trim a vascular graft subsequent to placement of the vascular graft in a vascular organ, which comprises:
a rotatable ring; and
a plurality of blades operatively connected to said rotatable ring and movable between an open position for receiving the vascular graft and a closed position for cutting the vascular graft upon rotation of the rotatable ring.

19. A graft cutter adapted to trim a vascular graft subsequent to placement of the vascular graft within a vascular organ, which comprises:
a lasso cutter including a sharp wire-like material and adapted for movement between an open configuration for receiving the vascular graft and a closed configuration for compressing against and thereby cutting the vascular graft.
